(19) United States
(12) Patent Application Publication
(10) Pub. No.: US 2008/0279903 A1
(43) Pub. Date: Nov. 13, 2008

(54) Edible thin film strips and process for making

(76) Inventor: Hwa-song Liu, Puyallup, WA (US)

Correspondence Address: COHEN, PONTANI, LIEBERMAN & PAVANE LLP, 551 FIFTH AVENUE, SUITE 1210, NEW YORK, NY, 10176, US

(21) Appl. No.: 12/151,463

(22) Filed: May 7, 2008

Related U.S. Application Data
(60) Provisional application No. 60/927,995, filed on May 7, 2007.

(51) Int. Cl. A61K 8/02; A61Q 11/00

(52) U.S. Cl. 424/401; 424/58

(57) ABSTRACT

A method to produce edible film and apply the method to produce products containing ingredients from natural sources in one film.

### RELATED APPLICATIONS

This application claims priority from U.S. Provisional Patent Application Ser. No. 60/927,995 which was filed on May 7, 2007.

### BACKGROUND OF THE INVENTION

1. Field of the Invention

The present invention relates to edible films containing an effective amount of active ingredients from natural sources, and methods of producing these films.

2. Description of the Related Art

Edible thin film product has been in the market for many years. It is a water-soluble, carbohydrate-based thin film that contains active ingredients. Most notable product on the market now is the Listerine edible film that contains Menthol as the active ingredient.

FIG. 1 illustrates the general procedures to produce edible thin film as follows:

1. Active ingredients are blended with melted film-forming material, such as cellulose, gelatin to produce an evenly distributed liquid.

2. The solution is then spread evenly in a plain surface to form a gel-like sheet.

3. The sheet is then dried using heat or vacuum evaporation to form a thin layer.

The limitation of the current edible thin-film technology is its capacity to hold higher volumes of active ingredients in one strip. Based on the size and constraints of the material, the current thin film technology can hold up to 50 mg of active ingredients in one single film, which is equivalent to 50% of its weight. This may be sufficient for pharmaceutical application. However, for nutraceutical products, especially product with ingredients derived from natural resources. The volume is too small to contain an effect dosage in one film.

Currently there is technology available to apply multiple-layer technology to increase the holding capacity of the thin film. By placing active ingredients sandwiched between two cover layers of thin film, the hold capacity of the thin film can increase up to over 200 mg, which is equivalent to 50˜65% of its weight. The multiple-layer structure of the film is depicted in FIG. 2.

However, even with the capacity to hold more than 200 mg on one single thin film, it is still not applicable for herbal extract to reach efficient dosing, as each application will in general require dosage in several grams. In most cases each nutraceutical product generally contains several active ingredients, with current technology it is impossible to develop product with sufficient dosage volume based on single thin film.

### SUMMARY OF THE INVENTION

One embodiment of my invention is to combine multiple-ingredient extraction and high-ratio condensation to produce uniform active ingredients composition applicable for use in thin films. The method is to begin by mixing raw herb materials and a predetermined preferred combination, then apply extraction to the raw herb. The extract then undergoes condensation to remove excessive solvent. The final condensed extract will go through stabilization process before blending with material to produce the thin film.

To produce an edible thin film with ingredient from natural resource that will contain high dose of active ingredient, we generally employ the following steps.

Instead of starting with active ingredients in powder form, we use raw herb in combined extraction process. This will ensure uniform distribution of active components in the ingredient and to optimize the quantity of ingredient to encapsulate into one film.

Prepare raw material in a predetermined combination. The formula include combination of herbal raw material documented in Traditional Chinese Medicine combination, herbal medicine and herbal material for dietary supplement ingredients.

If we know the active or key components of individual herb, we can use different analytical method, such as chromatography or spectroscopy instrumentation to identify the key components in the herbs to optimize the extraction yield.

Perform crude extraction. A crude extraction means decoction of the herb materials using water or mixture of alcohol and water. This will recover chemical constituents soluble to water and alcohol from the herbs. It is considered the most common extraction methodology for plant-based raw material. The purpose of this procedure is to determine the content of key components in the herbs as baseline for further refined extraction process.

Perform refined extraction methods to enhance extraction yield based on the baseline of the crude extraction. Several extractions methods can be applied for refined extraction method. They include but are not limited to: Organic solvent extraction, where an organic solvent, such as n-hexane, ethyl acetate . . . etc. is used; Supercritical extraction, which uses liquid state carbon dioxide in high pressure to extract the herb, is commonly used in extraction of volatile chemicals and essential oil; Microwave extraction, which applies microwave to excite molecules in raw material followed by extraction method to enhance solubility; Ultrasound cell-breaking extraction method, which uses ultrasound to break the cell wall of the molecule to release content inside plant cell; high-pressure quenching extraction, which use high pressure to squeeze the raw material mixture. Numerous methods are under development currently which can later be applied to enhance the extraction rate of the herbs.

Following the extraction, perform condensation procedure to remove excess solvent residue and to increase the concentration of the active components. The methods generally applied to condensation include but are not limited to: Vacuum evaporation, which use vacuum to physically remove solvent in the extract; Microwave evaporation, which use microwave to evaporate the water molecules and remove from the extract; Super-critical evaporation, which adjusts temperature and pressure at critical point to physically separate the solvent from the extract. Several new methods are in development and can be later applied to this process.

Compare the contents of the active components in the final extract to components in crude extract to calculate the efficacy rate of the refined extraction and condensation.

Mix refined extract with other active ingredients that is not botanical-based, such as Vitamin and other dietary supplement ingredients, such as glucosamine or melatonin. Blend thoroughly so that the mixtures are evenly distributed.

Stabilize the ingredients mixture before applying to the thin film. This is to ensure the stability of the active ingredients during the production of the film. The usual methodologies of stabilization include spray drying or granulation to form powder ingredients; addition of preservative to prevent bacteria contamination, radiation sanitation, freeze drying; freezing or introduction of inert gas in packaging.

The method can apply to any methodologies to produce edible thin film. The general procedure is to blend the active ingredient mixture with film-forming material to produce an evenly distributed solution. The solution is then spread evenly in a plain surface to form a gel-like sheet. The sheet is then dried using heat of vacuum evaporation to form a thin layer.

The application of this technology will cover herbal, mineral and vitamin, which are soluble by water, alcohol, oil or organic solvent. The intermediate from the extraction and concentration can be either paste or powder form.

FIG. 3 illustrates the procedures described above to produce single layer edible film that contains high concentration of ingredients from botanical source combined with other ingredients. To produce multiple film system containing high volume of ingredients from botanical source, we can make modification to the method used to produce single film to accommodate different compositions of the herbal combinations. Each procedure be can carried out separately depending on different physical characteristics in the process, the raw material can also be combined to perform certain procedures. The selection of different methods is to optimize the yield of the active components in the final product.

Prepare the botanical material in different categories based on different characteristics of the raw material and parameters involved in the extraction and condensation processes.

The characteristics of the raw material for the classification normally include:
a. General practice as described in traditional documentation
b. Target chemical components in individual herb
c. Solubility of individual herb to different solvent.
d. polarity of the chemical extract
e. extraction condition
f. Stability of different extracts to time, temperature and solvent.
g. The efficacy of concentration process to different raw material

Based on the choice of the herb combination in different categories, we can use different extraction methods to enhance the extraction efficacy.

At each step of the procedure we can determine whether we would perform condensation and stabilization separately, or we can combine certain procedures so that we can optimize the yield of the active components in different herbs.

Once we have created the active ingredients following stabilization procedures, there are several methods to prepare multi-layer films
a. Mix all active ingredients with raw material to form edible thin film, and prepare first layer of thin film on flat surface, let dry and then cover with the second layer of thin layer with the same material, continue until we reach the desired number of thin layers; or
b. Prepare single thin films for different combinations of ingredients, then combine the single thin films to produce multiple layer thin film.

By applying different technology to enhance extraction, we can substantially improve the yield of extraction. In this way we can condense the active components in herb extract to over 30 times greater than its normal volume, By combining the available technology of multiple layer system, we can achieve the holding capacity of active ingredient on one thin film that exceeds current capacity available. Combine with the technique to hold active ingredient at the capacity of 50%˜65% of the weight of its weight and the technique to condense the herb extract to over 30 times greater than its normal volume. Accordingly, we can hold active ingredient extracted from raw herb in the volume which is 10 to 20 times in weight ratio of a single film.

### BRIEF DESCRIPTION OF THE DRAWINGS

In the drawings:

FIG. 1 is a flow diagram of the general procedure to produce edible thin films.

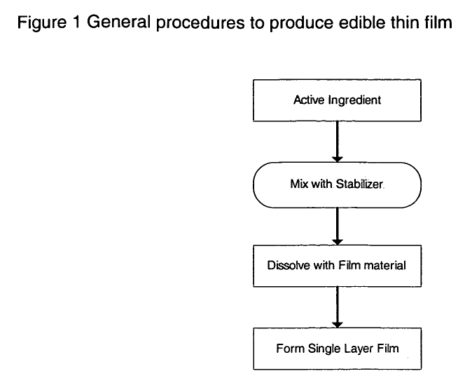

FIG. 2 depicts schematically the structure of a multilayer thin film.

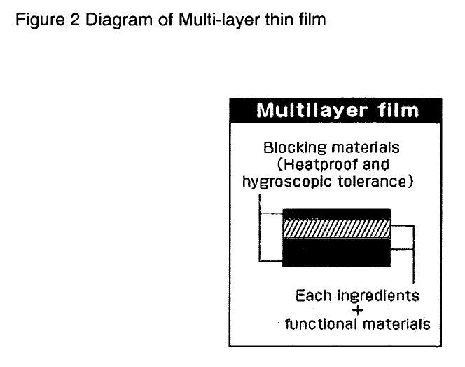

FIG. 3 is a flow diagram of the steps to produce a single film containing a large volume of active ingredients extracted from natural resources.

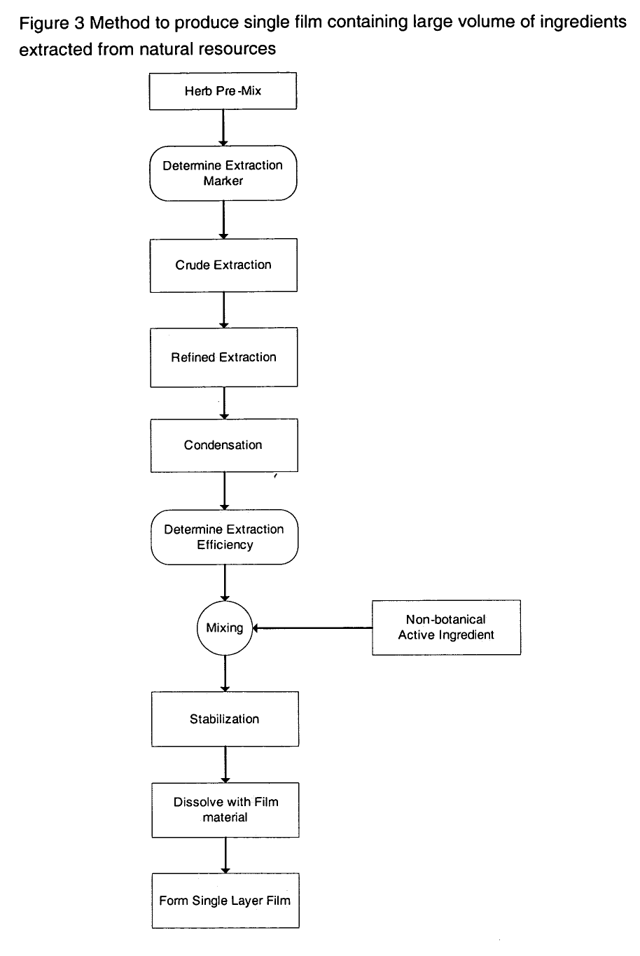

FIG. 4 is a flow diagram of the steps in producing a multiple-layer film containing large volume of active ingredients extracted from natural sources.

FIG. 5 is a flow diagram of the steps in producing a multi-layer thin film.

### DETAILED DESCRIPTION OF THE PRESENTLY PREFERRED EMBODIMENTS

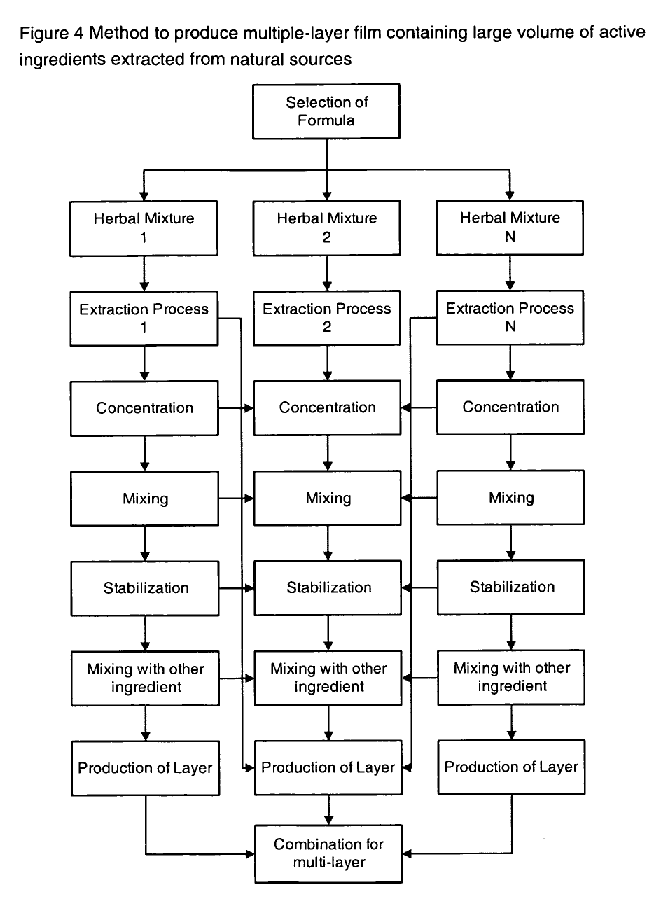

FIG. 4 illustrates one embodiment of the present inventive methodology to produce multiple edible thin films containing high volume of ingredients from botanical sources.

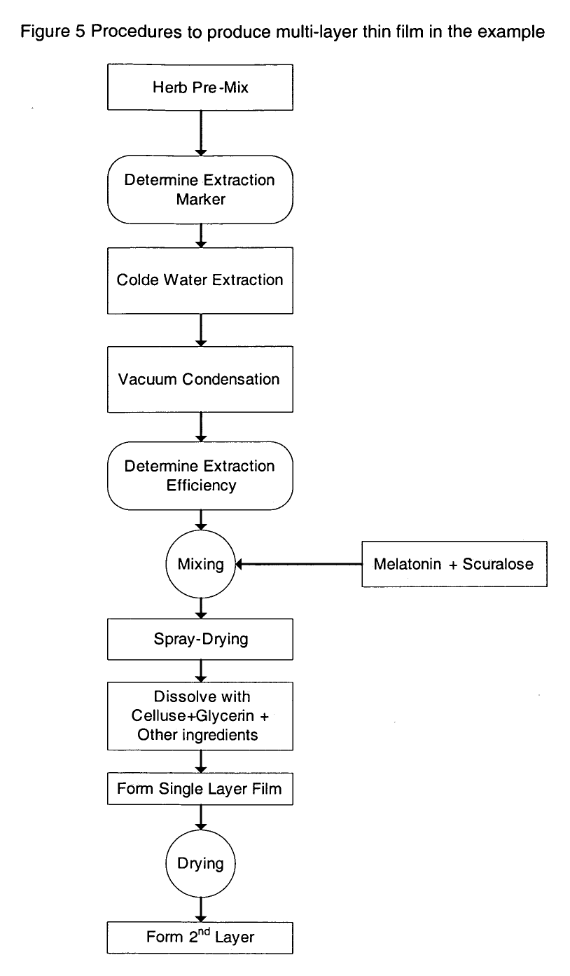

Based on the methodology, we have successfully produced a sample which consists of several ingredients from natural resources, and is useful when administered to a person to induce sleep. The total thickness of the thin film is over 100 μm, which consists of two thin layers. In combination with other ingredients, the total amount of the active ingredients has exceeded 800 mg. FIG. 5 describes the procedure flowchart for producing the thin film.

The procedure of the application includes:

1. The products contain active ingredients from botanical source in fixed combination of Chinese lovage rhizome, Poria, Jujube seed, Anemarrhenea rhizome, Licorice root and Momordicae fruit. The formula is based on traditional Chinese medicinal formula “Suan-Zao-Ren Tang”.

2. The product also contains other active ingredients such as melatonin and sucralose.

3. The raw herbs in botanical formula are first mixed to undergo extraction. Two active components are selected to determine the extraction efficacy. The components are Ferulic acid and Glycyrrhizin. In order to reduce the extraction of polysaccharide in the process, a cold water extraction is performed on extended time. The temperature is set between 35˜75 degree Celsius, while the extraction time is set to 4˜6 hours.

4. The extract is then to go through vacuum extraction to remove solvent.

5. The condensed extract is then mixed with a fixed amount of melatonin which is then followed by spray drying to produce stabilized powder mixture.

6. The mixture is then mixed with molten cellulose, glycetin, which is then added with flavoring and sweetener to form a uniform gel mixture.

7. The gel mixture is then spread on a flat surface to form a uniform thin layer of liquid gel. Then the thin layer is cooled to form a solid thin layer.

8. The second layer of mixture is spread on top of the first layer then dried as described in Step 7. This is to increase the content of the active ingredients to desired concentration of nearly 800 mg. In this process the thin layer will be less susceptible to breakage.

9. The final product has an active ingredient capacity of nearly 800 mg of botanical raw herbs combined in 40 mg of extract. The extraction efficacy is 20×. It also contains non-botanical herbs which is about 10 mg.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

I claim:

1. An edible thin film containing at least one active ingredient that is extracted and concentrated from a natural source.

2. The film according to claim 1 wherein the active ingredient is up to about 30 times more concentrated than the active ingredient is present in the natural source.

3. The film according to claim 1 wherein the active ingredients are extracted and concentrated from natural sources comprising Chinese lovage rhizome, Poria, Jujube seed, Anemarrhenea rhizome, Licorice root and Momordicae fruit.

4. A method of producing an edible thin film comprising extracting active ingredients from natural sources, concentrating the extracts, and forming the concentrated extracts into a thin film.

5. The method of claim 4 wherein the natural sources comprises raw herbs.

6. The method of claim 4 wherein the natural sources comprises Chinese lovage rhizome, Poria, Jujube seed, Anemarrhenea rhizome, Licorice root and Momordicae fruit.

7. The method of claim 4 which further comprises the step of stabilizing the active ingredients.

8. The method of claim 4 wherein the concentrated extracts are formed into two or more layers.

9. The method of claim 4 wherein the active ingredients is from 80% to 100% of the weight of the thin film.

10. The method of claim 4 which further comprises the step of adding active ingredients from non-natural sources to an active ingredient from a natural source.
